(19) United States
(12) Patent Application Publication
(10) Pub. No.: US 2023/0404863 A1
(43) Pub. Date: Dec. 21, 2023

(54) MICRONEEDLE PATCH AND PREPARATION METHOD THEREFOR

(71) Applicant: SUZHOU REVEDA MEDICAL BIOTECH CO., LTD., Suzhou (CN)

(72) Inventors: Bing LI, Suzhou (CN); Qinghui JIANG, Suzhou (CN); Nan ZHOU, Suzhou (CN); Ping YAN, Suzhou (CN)

(21) Appl. No.: 18/253,270

(22) PCT Filed: Jun. 24, 2021
(86) PCT No.: PCT/CN2021/102217

§ 371 Date: May 17, 2023

(30) Foreign Application Priority Data
Dec. 31, 2020 (CN) 202011632383.6

(51) Int. Cl. A61K 8/02; A61K 8/35; A61K 8/73; A61K 8/65; A61Q 19/08; A61M 37/00; A45D 44/00

(52) U.S. Cl. CPC A61K 8/0216; A61K 8/35; A61K 8/738; A61K 8/65; A61K 8/735; A61Q 19/08; A61M 37/0015; A45D 44/002; A61M 2037/0023; A61M 2037/0053

(57) ABSTRACT

A microneedle patch and a preparation method therefor are disclosed. The microneedle patch includes a base and a number of needles arranged on a surface of the base. The needles are dissolvable pointed bodies and contain a composition with cosmetic effects. The composition includes astaxanthin, hydrophilically modified resveratrol, collagen and hyaluromic acid. The method includes the steps of: pouring an aqueous solution of the composition into a mold; vacuumizing the mold; removing water therefrom; and demoulding the mold, thereby obtaining the microneedle patch. The present invention provides a variety of advantages including high safety, high stability, good absorption into the skin and obvious cosmetic effects.

### TECHNICAL FIELD

The present invention pertains to the field of medical devices and, in particular, relates to a component for introducing a medium into the body through skin penetration and a preparation method therefor.

### BACKGROUND

The skin gradually ages with age. Skin aging that occurs due to genetic and irresistible factors (gravity, deterioration of physiological functions of vital organs, etc.) and is not or less affected by external stimuli is called natural aging. In addition to being affected by age, aging is directly related to sunlight exposure. Aging that occurs mainly due to the influence of sunlight including ultraviolet (UV) rays is called photoaging. As a combined result of natural aging and UV exposure, photoaging is skin damage caused by long-term sunlight exposure. When the skin is exposed to sunlight, UV radiation therein is able to penetrate 4-5 cm into the skin and form various free radicals, which can hinder the synthesis of collagen, making the whole face look less full. UV radiation can kill our skin cells or cause DNA damage therein, making them unable to rejuvenate. Dermatologic manifestations of exposure to UV radiation may include roughness, thickening, dryness, sag, deepened or pronounced wrinkles, excessive local pigmentation or telangiectasia, and even various benign or malignant tumors (e.g., actinic keratosis, squamous cell carcinoma, malignant melanoma, etc.). The face, neck and the extensor sides of the upper limbs are most susceptible to photoaging, and aging can be reduced by about 80% when good protection against light exposure is attained.

Cosmetics and surgery are two common cosmetology approaches for facial beauty. As one of the common approaches, cosmetics for skin care available on the market are mainly liquids, lotions, ointments, creams, facial masks and the like. Facial masks can be principally categorized into wash-off masks and sheet masks. A wash-off mask is typically made of a pasty or creamy substance, and its use is tedious and cumbersome because it requires application of the substance to the face. Moreover, when an excessive amount of the creamy substance is applied, pores may be clogged up, possibly leading to the development of fat granules, closed comedones or even acne. A sheet mask is, for example, a piece of non-woven fabric soaked with active ingredients. However, due to a limited carrying capacity of the non-woven fabric, a large amount of the active ingredients remains in the package, leading to a significant reduction in utilization of the active ingredients. Further, both these two types of facial masks only remain in contact with and cannot penetrate the stratum corneum despite the fact that the active ingredients can exert their effects only when delivered through the stratum corneum barrier. Thus, absorption of the active ingredients is very poor.

Surgical cosmetology is typically accomplished by, among others, injection of hyaluronic acid, botulinum toxin or snake toxin, face lift, radio frequency (RF) or laser treatment, and is associated with some technical difficulties and risks. In practice, such surgical procedures must be carried out in specialized institutions, limiting the application of this approach. Moreover, these procedures are risky, expensive and require a long recovery period. Since photoaging is a long-lasting, continuous external cause of skin damage, surgical results seldom last for long.

### SUMMARY OF THE INVENTION

The present invention provides a microneedle patch and a method of preparation thereof, which overcome the problems of instability of active ingredients, a limited amount thereof that can be absorbed into the skin and unsatisfactory cosmetic effects associated with the existing anti-photoaging, repair and anti-aging patches.

To this end, the microneedle patch of the present invention includes a base and a number of needles arranged on a surface of the base. The needles are dissolvable pointed bodies and contain a composition with cosmetic effects. The composition includes astaxanthin, hydrophilically modified resveratrol, collagen and hyaluromic acid.

Astaxanthin and resveratrol are unstable and will easily lose their activities. Consequently, they may fail to exert their effects when used in anti-photoaging skin care products. In order to overcome this problem, according to the present invention, hydrophilically modified resveratrol is mixed with astaxanthin, collagen and hyaluromic acid, and the mixture is used to fabricate microneedles. In this way, the active ingredients are effectively sealed and isolated from the outside world. Moreover, the combined use of astaxanthin, resveratrol, collagen and hyaluromic acid can provide synergistic anti-photoaging, repair and anti-aging effects to the skin.

When needles in the microneedle patch are inserted into the skin, they will dissolve, swell or break therein, and the active ingredients will be absorbed by the skin cells, providing damage repair and resistance to damage that may be caused by ambient light. In this way, cosmetic effects can be delivered in a short time.

The hydrophilically modified resveratrol is better compatible with astaxanthin and can better permeate through the skin. Therefore, in addition to increased percutaneous absorption, the microneedles enable a higher absorption rate of the functional ingredient.

Optionally, the hydrophilically modified resveratrol may be an inclusion compound formed by molecules of hydroxypropyl-β-cyclodextrin and resveratrol.

Optionally, a mass ratio of the resveratrol to the hydroxypropyl-β-cyclodextrin may be (0.8-1.2):(9-12).

Optionally, weight parts of the components in the composition may be as follows:
Astaxanthin 0.05-0.3 parts;
Hydrophilically modified resveratrol 0.1-0.3 parts;
Collagen 1.5-5 parts;
Hyaluromic acid 70-95 parts.

Optionally, the microneedle patch may have a total height of 200 μm to 500 μm, and the needles may have a height of 30 μm to 350 μm and a maximum extent of 100 μm to 400 μm at their joints with the base.

Such precisely designed and controlled dimensions enable the microneedles to reach between the epidermis and dermis of the skin. Thus, the needles can be inserted into the skin without causing pain or bleeding. Moreover, the active ingredients can be concentrated at a target site where they are intended to exert their effects. Further, the holes left by the needles can close quickly.

Optionally, the needles may be arranged on the surface of the base into an array with a density of 50-300 needles per cm².

Optionally, the microneedle patch may be used around the eyes or on the face, and the cosmetic effects may include anti-photoaging effects.

The present invention also provides a method of preparing the microneedle patch as defined above, which includes the steps of: pouring an aqueous solution of the composition into a mold; vacuumizing the mold; removing water therefrom; and demoulding the mold, thereby obtaining the microneedle patch.

Optionally, the aqueous solution of the composition may be prepared using a process including: dissolving hyaluromic acid or a salt thereof into water; and then adding astaxanthin, the hydrophilically modified resveratrol and collagen thereto and homogeneously stirring the mixture.

Optionally, the needles may be cones, quadrangular pyramids or triangular pyramids.

The present invention also provides a method of preparing a microneedle patch, which includes the steps of:
obtaining hydrophilically modified resveratrol by adding a solution of resveratrol in ethanol to an aqueous solution of hydroxypropyl-β-cyclodextrin and homogeneously stirring, filtering and drying the mixture;
forming an aqueous solution of a composition by successively adding hyaluromic acid, astaxanthin, the hydrophilically modified resveratrol and collagen to water and homogeneously stirring the mixture; and
obtaining the microneedle patch by pouring the aqueous solution of the composition into a mold, vacuumizing the mold, removing water therefrom, and demoulding the mold.

Preferably, a mass ratio of the resveratrol to the hydroxypropyl-β-cyclodextrin may be (0.8-1.2):(9-12).

Optionally, weight parts of the components in the composition may be as follows:
Astaxanthin 0.05-0.3 parts;
Hydrophilically modified resveratrol 0.1-0.3 parts;
Collagen 1.5-5 parts;
Hyaluromic acid 70-95 parts.

Preferably, the microneedle patch may have a total height of 200 μm to 500 μm, and the needles may have a height of 30 μm to 350 μm and a maximum extent of 100 μm to 400 μm at their joints with the base.

Preferably, the needles may be cones, quadrangular pyramids or triangular pyramids.

According to the present invention, a water-soluble microneedle patch is prepared from active ingredients with cosmetic effects. The microneedle patch not only significantly enhances stability of the active ingredients in the composition, but also ensures that microneedles therein have sufficient mechanical strength that allows them to penetrate the stratum corneum to form micro-channels for transport of the active ingredients to a subcutaneous site to deliver anti-photoaging, repair and anti-aging effects there without any toxic or side effect. The active ingredients can act synergistically and have good water solubility. Compared to the existing anti-photoaging patches, the present invention has a variety of advantages including high safety, high stability, good absorption into the skin and obvious cosmetic effects.

### BRIEF DESCRIPTION OF THE DRAWINGS

FIG. 1 is a schematic diagram showing the structure of microneedle patches prepared in Embodiments 1 to 3.

FIG. 2 shows the results of cytotoxicity tests performed on the microneedle patches in Embodiment 4.

FIG. 3 shows photos of an aluminum foil taken in a penetration ability test in Embodiment 5, in which a is a photo of a front side of the aluminum foil; b is a photo of a back side of the aluminum foil; c is a photo of the front side of the aluminum foil that has been penetrated by a microneedle patch; and d is a photo of the back side of the aluminum foil that has been penetrated by the microneedle patch.

FIG. 4 shows photos of fresh porcine skin taken in a penetration ability test in Embodiment 5, in which a is a photo of the fresh porcine skin; b is a photo of the porcine skin on which a microneedle patch is pressed; and c is a photo of the porcine skin after the microneedle patch is removed.

FIG. 5 shows a comparison between stability of hydrophilically modified resveratrol in a patch and that in an aqueous solution in Embodiment 6, in which a shows spectra of resveratrol in the patch at Day 0 and Day 7 measured on a UV spectrophotometer, and b shows spectra of resveratrol in the aqueous solution at Day 0 and Day 7 measured on a UV spectrophotometer.

FIG. 6 shows a comparison between stability of astaxanthin in a patch and that in an aqueous solution in Embodiment 6, in which a shows spectra of astaxanthin in the patch at Day 0 and Day 6 measured on a UV spectrophotometer, and b shows spectra of astaxanthin in the aqueous solution at Day 0 and Day 6 measured on a UV spectrophotometer.

FIG. 7 shows photos showing color comparisons between astaxanthin in a patch and astaxanthin in an aqueous solution in Embodiment 6, in which a shows a color comparison therebetween at Day 0, and b shows a color comparison therebetween at Day 6.

In these figures,
10-base; 20-needle.

### DETAILED DESCRIPTION

For ease of understanding, the microneedle patch and the method of preparation thereof are described below by way of examples. It is to be understood that these examples are presented merely for the purpose of illustrating the present invention and not intended to limit the scope thereof in any sense.

The microneedle patch proposed in the present invention is generally used for anti-aging and cosmetic care of skin exposed to sunlight, such as face skin, skin around the eyes, neck skin, hand skin, etc., and may be designed in various shapes and sizes to meet different needs.

Unless otherwise specified, in the examples, all the raw materials and instruments used are commercially available conventional products, and all the technical means and processes employed are conventional means and processes.

### EMBODIMENTS 1 TO 3

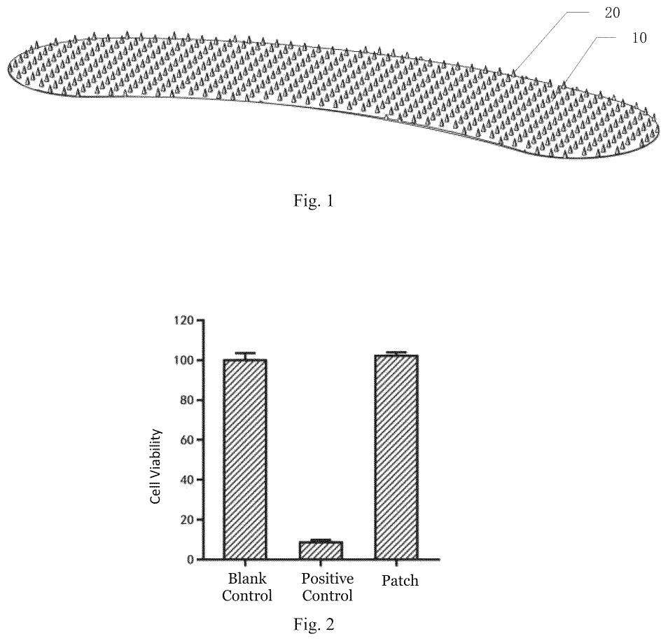

As shown in FIG. 1, as an example, the microneedle patch may be structured for use on skin around the eyes. It includes a base 10 and a plurality of needles 20 arranged into an array on a surface of the base 10. In this embodiment, the needles are solid cones. Of course, they may also be implemented as quadrangular pyramids, triangular pyramids or higher-order pyramids. Alternatively, they may be hollow pointed bodies.

The microneedle patch has a total height of 200-500 μm, and the needles have a height of 30-350 μm. The needles have a maximum extent of 100-400 μm at their joints with the base (e.g., a diameter of bases in case of conical needles) and are arranged with a density of 50-300 needles/cm². The total height of the microneedle patch refers to a straight-line distance from a bottom surface of the base to apices of the needles. The height of the needles refers to a distance measured between the bases and apices of the needles.

Dimensions of microneedle patches prepared in Embodiments 1 to 3 are summarized in Table 1.

TABLE 1

| | | | Diameter | |
|---|---|---|---|---|
| | Total | Height of | of Bases | |
| | Height | Needles | of needles | Density |
| | (μm) | (μm) | (μm) | (needles/cm²) |
| Embodiment 1 | 500 | 300 | 400 | 50 |
| Embodiment 2 | 300 | 150 | 250 | 200 |
| Embodiment 3 | 200 | 30 | 100 | 300 |

Each of the microneedle patches was prepared according to a method including the steps as follows.

1) Prepare hydrophilically modified resveratrol. A proper amount of hydroxypropyl-β-cyclodextrin (a purchased mixture) was weight and dissolved in water, producing a solution with a concentration of 0.1 g/mL. 0.1 g of resveratrol was weight and dissolved in a proper amount of anhydrous ethanol. The solution was slowly added to the aqueous solution of cyclodextrin at 30° C. under magnetic stirring. A mass ratio of resveratrol to hydroxypropyl-β-cyclodextrin was controlled at (0.8-1.2):(9-12). After being stirred for 2 hours, the mixture was filtered to remove impurities and then freeze-dried. The product was resveratrol that had been modified to water-soluble. In the prepared hydrophilically modified resveratrol, molecules of hydroxypropyl-β-cyclodextrin and resveratrol formed an inclusion compound.

2) Prepare a composition solution. Hyaluromic acid was dissolved in water, and stirred for about 0.5 hours, resulting in a clear, homogeneous solution. Astaxanthin, the hydrophilically modified resveratrol prepared in step 1) and collagen were successively added to the solution and homogenized therein by stirring. Astaxanthin, the hydrophilically modified resveratrol, collagen and hyaluromic acid were present at the following mass ratio:

| | | |
|---|---|---|
| Astaxanthin | 0.05-0.3 | parts |
| Hydrophilically modified resveratrol | 0.1-0.3 | parts |
| Collagen | 1.5-5 | parts |
| Hyaluromic acid | 70-95 | parts |

3) Form the microneedle patch. The composition solution prepared in step 2) was poured into a microneedle mold, and the mold was then vacuumized and dried for removal of water. The microneedle patch was obtained after the mold was removed.

The mass ratios of the components and axial pressures per needle in the microneedle patches prepared in Embodiments 1 to 3 are presented in Table 2. As shown in Table 2, the microneedles exhibit high mechanical strength. The axial pressures per needle were measured by pressing needles with a flat pressure head and recording axial forces at different displacements. The flat pressure head has an effective area of 0.25 cm² encompassing about 25 needles. The axial pressures per needle are calculated from recorded axial forces at a displacement of 0.2 mm

TABLE 2

| | Mass Ratio of | | | | | Axial |
|---|---|---|---|---|---|---|
| | Resveratrol to | | Hydrophilically | | | Pressure/ |
| | Hydroxypropyl- | | Modified | | Hyaluromic | Needle |
| | β-Cyclodextrin | Astaxanthin | Resveratrol | Collagen | Acid | (N/0.2 mm) |
| Embodiment 1 | 0.8:12 | 0.05 | 0.1 | 1.5 | 98.35 | 1.9 |
| Embodiment 2 | 1.0:11 | 0.1 | 0.2 | 2 | 97.7 | 1.8 |
| Embodiment 3 | 1.2:9 | 0.3 | 0.3 | 5 | 94.4 | 1.8 |

### EMBODIMENT 4: CYTOTOXICITY

Cytotoxicity of a microneedle patch prepared in accordance with Embodiment 3 was tested.

The microneedle patch of Embodiment 3, which was 0.12-mm thick and weighed 400 mg, was dissolved in 4 mL of cell culture medium. Cytotoxicity of the microneedle patch was tested on mouse fibroblasts (L929 cells). As shown in FIG. 2, liquid culture medium of the same batch was used as a blank control, and a 14% DMSO solution as a positive control. The patch was the solution of the microneedle patch of Embodiment 3. The results show that the microneedle patch is completely non-toxic and can be safely used for absorption into the skin.

### EMBODIMENT 5: PENETRATION ABILITY

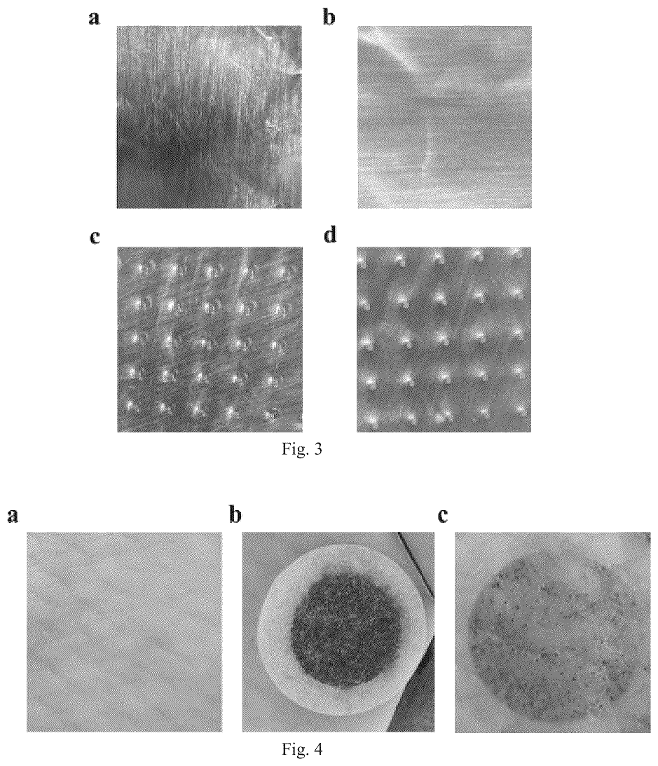

A microneedle patch prepared in accordance with Embodiment 3 was cut into a round shape with a diameter of 1 cm and pressed on an aluminum foil for 30 s. The results were observed and recorded. As shown in FIG. 3, the aluminum foil was penetrated, demonstrating a certain level of mechanical strength of the mechanical strength of the anti-photoaging microneedle patch of the present invention.

A microneedle patch prepared in accordance with Embodiment 3 was dyed with methylene blue, cut into a round shape with a diameter of 1 cm, pressed for 40 s on fresh porcine skin that has been processed to remove subcutaneous fat therefrom, and then removed. As shown in FIG. 4, staining of the porcine skin was observed, and it was found after the removal of the microneedle patch that the porcine skin was penetrated, demonstrating that the microneedle patch of the present invention is mechanically strong enough to penetrate the skin.

### EMBODIMENT 6: STABILITY

Astaxanthin and resveratrol are easily oxidized and are very unstable in water and in emulsions. Due to immiscibility of resveratrol with water, it was first hydrophilically modified, and its stability in patches and that in solutions was compared. The patches were made of astaxanthin and the hydrophilically modified resveratrol, and the solutions were aqueous solutions of astaxanthin and the hydrophilically modified resveratrol at the same concentrations. Compositions of the patches used in stability tests are shown in Table 3.

TABLE 3

| | | Hydrophilically | |
|---|---|---|---|
| Components/Mass | | Modified | Hyaluromic |
| Ratio | Astaxanthin | Resveratrol | Acid |
| Patch Sample 1 | 0% | 0.2% | 99.8% |
| Patch Sample 2 | 1% | 0% | 99% |

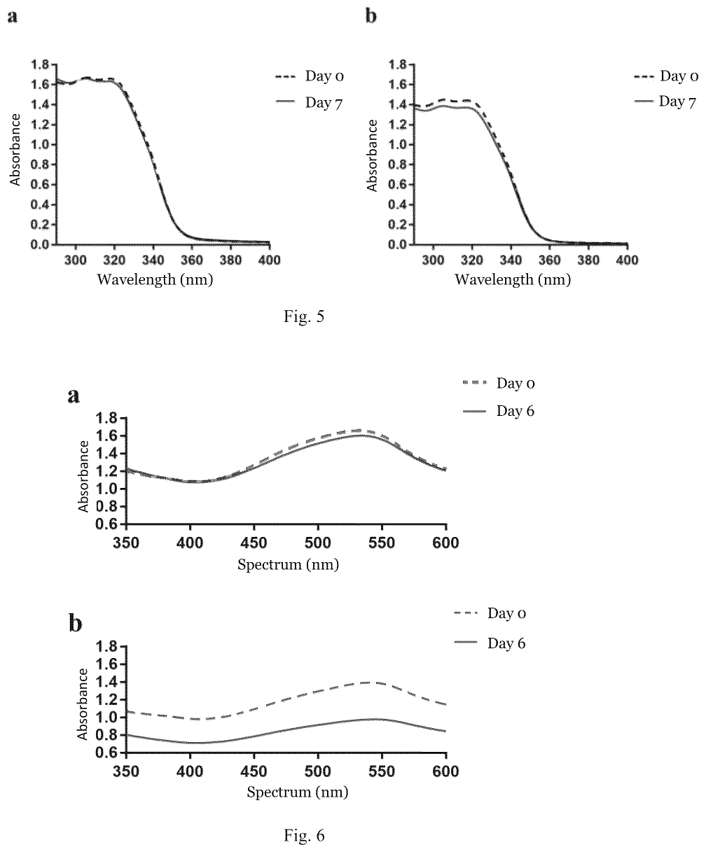

As shown in FIG. 5, Patch Sample 1 was left at room temperature for 7 days without avoiding exposure to light, and the detection results of a UV spectrophotometer showed that there was no significant change between the content of resveratrol at the beginning and that after 7 days. In contrast, under the same conditions, the content of the hydrophilically modified resveratrol after 7 days was lower than that at the beginning. This indicates that the stability of the active ingredient resveratrol in Patch Sample 1 is superior over that in the aqueous solution.

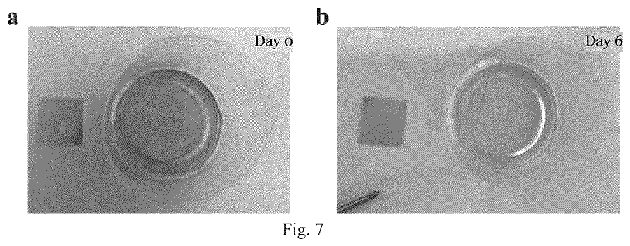

As shown in FIG. 6, Patch Sample 2 was left at room temperature for 6 days without avoiding exposure to light, and the detection results of a UV spectrophotometer showed that there was no significant change between the content of astaxanthin at the beginning and that after 6 days. In contrast, under the same conditions, the content of astaxanthin after 6 days was significantly lower than that at the beginning. Moreover, as shown in FIG. 7, after being left at room temperature for 6 days, the color of astaxanthin in the solution became much lighter, while the color of astaxanthin in the patch did not show a significant change. This indicates that the stability of astaxanthin in Patch Sample 2 is superior over that in the aqueous solution.

At last, it is to be noted that the above embodiments are presented merely for the purpose of illustrating the subject matter of the present invention and are not intended to limit it in any sense. Although the present invention has been described in detail with reference to the foregoing embodiments, those of ordinary skill in the art will appreciate that modifications to the technical solutions provided in the embodiments or equivalents of part or all of the features thereof are still possible and such modifications or equivalents do not deviate the essence of the technical solutions from the scope of the various embodiments of the invention.

1. A microneedle patch, comprising a base and a number of needles arranged on a surface of the base, wherein the needles are dissolvable pointed bodies, the needles containing a composition with cosmetic effects, the composition comprising astaxanthin, hydrophilically modified resveratrol, collagen and hyaluromic acid.

2. The microneedle patch according to claim 1, wherein the hydrophilically modified resveratrol is an inclusion compound formed by molecules of hydroxypropyl-β-cyclodextrin and resveratrol.

3. The microneedle patch according to claim 2, wherein a mass ratio of the resveratrol to the hydroxypropyl-β-cyclodextrin is (0.8-1.2):(9-12).

4. The microneedle patch according to claim 1, wherein weight parts of the components in the composition are as follows:
Astaxanthin 0.05-0.3 parts;
Hydrophilically modified resveratrol 0.1-0.3 parts;
Collagen 1.5-5 parts;
Hyaluromic acid 70-95 parts.

5. The microneedle patch according to claim 1, wherein the microneedle patch has a total height of 200 μm to 500 μm, the needles have a height of 30 μm to 350 μm, and the size at a widest point where the needles joint with the base is 100 μm to 400 μm.

6. The microneedle patch according to claim 1, wherein the needles are arranged on the surface of the base into an array, the array having a density of 50-300 needles per cm².

7. The microneedle patch according to any one of claims 1 to 6, wherein the microneedle patch is used around the eyes or on the face, the cosmetic effects comprising anti-photoaging effects.

8. The microneedle patch according to claim 1, wherein the needles are cones, quadrangular pyramids or triangular pyramids.

9. A method of preparing a microneedle patch, comprising the steps of:
obtaining hydrophilically modified resveratrol by adding a solution of resveratrol in ethanol to an aqueous solution of hydroxypropyl-β-cyclodextrin and homogeneously stirring, filtering and drying the mixture;
forming an aqueous solution of a composition by successively adding hyaluromic acid, astaxanthin, the hydrophilically modified resveratrol and collagen to water and homogeneously stirring the mixture; and
obtaining the microneedle patch by pouring the aqueous solution of the composition into a mold, vacuumizing the mold, removing water therefrom, and demoulding the mold.

10. The method according to claim 9, wherein a mass ratio of the resveratrol to the hydroxypropyl-β-cyclodextrin is (0.8-1.2):(9-12).

11. The method according to claim 9, wherein weight parts of the components in the composition are as follows:
Astaxanthin 0.05-0.3 parts;
Hydrophilically modified resveratrol 0.1-0.3 parts;
Collagen 1.5-5 parts;
Hyaluromic acid 70-95 parts.

12. The method according to claim 9, wherein the microneedle patch has a total height of 200 μm to 500 μm, the needles have a height of 30 μm to 350 μm, and the size at a widest point where the needles joint with the base is 100 μm to 400 μm.

13. The method according to claim 9, wherein the needles are cones, quadrangular pyramids or triangular pyramids.
